(19) United States
(12) Patent Application Publication
(10) Pub. No.: US 2010/0121441 A1
(43) Pub. Date: May 13, 2010

(54) Surgical implant and method

(76) Inventors: Nabil Jabbour, Morgantown, WV (US); Jed Jabbour, Chicago, IL (US); Michael Gaynon, Palo Alto, CA (US)

Correspondence Address: A & G Intellectual Property Group, LLC, PO Box 4254, Morgantown, WV, 26504, US

(21) Appl. No.: 12/590,563

(22) Filed: Nov. 10, 2009

Related U.S. Application Data
(60) Provisional application No. 61/198,881, filed on Nov. 10, 2008.

(51) Int. Cl. A61F 2/14; A61N 1/30

(52) U.S. Cl. 623/4.1; 607/3; 604/20

(57) ABSTRACT

A first embodiment is a method comprising a bipolar diathermy for the eye wherein an electrode is connected to the ground electrode of the diathermy circuit, through a speculum which is applied between the eyelids, the electrode is then applied to the desired parts of the eye to create changes in layers of the eye using a designated, co-axial delivery and return path from the eye where the desired part of the eye can be the sclera, trans-sclera, and the trans-conjunctiva and the electrode can be a Jabbour electrode. The Jabbour electrode and a refillable implant are also disclosed in embodiments as well as a method of implantation for the implant.

### CROSS-REFERENCE TO RELATED APPLICATIONS

This application claims priority from provisional patent application numbered 61/198,881 filed on Nov. 10, 2008.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

1. Field of Invention

This invention relates to a surgical implant and method of implanting the device within the eye.

2. Description of the Prior Art

The delivery of therapeutic pharmacologic agents into the eye is severely limited by existing natural barriers. Drugs administered systemically ultimately reach a certain concentration in the blood stream and are, as such, distributed to the body with two main exceptions: the eye and the brain. The blood ocular barrier and the blood brain barrier are the reason for the minimal or inexistent penetration into the eye and the brain respectively. Furthermore, the use of drops, ointments, and even periocular injections usually result in low penetration and poorly sustained levels of drug deep into the eye.

To bypass such barriers, direct injections into the eye have been utilized with major limitations. In addition to the significant risk of infection and bleeding, intraocular injections achieve high but unsustainable and uneven levels of drug inside the eye with rapid drop in concentration within days from injection. Repeated injections then become necessary to maintain a therapeutic level. To maintain therapeutic levels of drug concentration it is necessary to repeat the injections almost weekly. This practice is totally impractical, highly risky, and very expensive.

Recently, several pharmacologic agents have been shown to be effective against some of the most chronic and devastating intraocular disorders. These disorders include diabetic retinopathy, vein occlusion, and macular degeneration responsible for most of the permanent visual loss seen in developed countries. Currently, the only two effective ways of delivering such treatment have been repeated intraocular injections in the order of every 4 to 6 weeks for at least 2 years, or the use of a slow release implant sutured internally to the pars plana and requiring invasive high risk surgery which presents additional further risks at the time of insertion, while the implant is in the eye and after it needs to be replaced.

Many patents have been issued in the field of surgical implants and methods for implantation. Included in these are U.S. Pat. Nos. 5,824,072, 6,331,313, 6,964,781, 4,300,557, 5,902,598, and 6,397,849. However, none of these patents disclose the novel improvements herein.

### BRIEF SUMMARY OF THE INVENTION

A first embodiment details a method to overcome the limitations of ocular barriers to deliver drugs at high penetration and sustained intraocular levels. The method can be performed in the perisclera, subretinal, intraocularly or to facilitate the penetration of drops.

It is an object of an embodiment for the periscleral systems that can be comprised of episcleral implants, intrascleral implants, and transcleral implants. Further the inventive method of the episcleral and intrascleral implants are comprised of implanting the implant within a pocket created within the eye.

It is another object of an embodiment to provide for a non-biodegradable, refillable ocular implant comprised of a semi-porous polymer able to be loaded with a drug, wherein the ocular implant is shaped to the desired area of implant and a water impermeable coating applied to portions of the implant not in contact with the desired area of implantation.

Another object an embodiment can provide for a surgical technique to create a pocket within the eye for episcleral implants.

A further object can provide for a subretinal technique comprising of a transvitreal needle subretinal delivery, traditional Pars plana vitrectomy (PPV) approach for subretinal implantation, and posterior approach after laser pre-treatment.

Another aspect of an embodiment can be an intraocular depot wherein a liquid depot semi-solid or a gel at 37° C. sinks to the bottom of the vitreous.

A further aspect can be the use of diathermy in a designated return path to create a closed and predictable circuit allowing for a safe retinal burn and subsequent chorioretinal scarring, which facilitates the transfer of drugs, locally from outside the intact scleral or the partially dissected sclera

### BRIEF DESCRIPTION OF THE SEVERAL VIEWS OF THE DRAWING

FIG. 1 is a view of an insulated pressure pump implant for protein-based drugs.

FIG. 2 is a view of the inter-scleral refillable system.

FIG. 3 is a view of the self retained, refillable transcleral implant.

FIG. 4 is a view of the suture guided transcleral refillable implant.

FIG. 5 is a view of the suture guided transcleral implant continued.

FIG. 6 is a view of the subretinal implant.

FIG. 7 is a view of the PPV subretinal implant approach.

FIG. 8 is a view of the posterior scleral approach.

FIG. 9 is a view of designated path diathermy.

FIG. 10 is a view of quadrant filled with applications in trans-conjunctival diathermy.

### DETAILED DESCRIPTION OF THE INVENTION

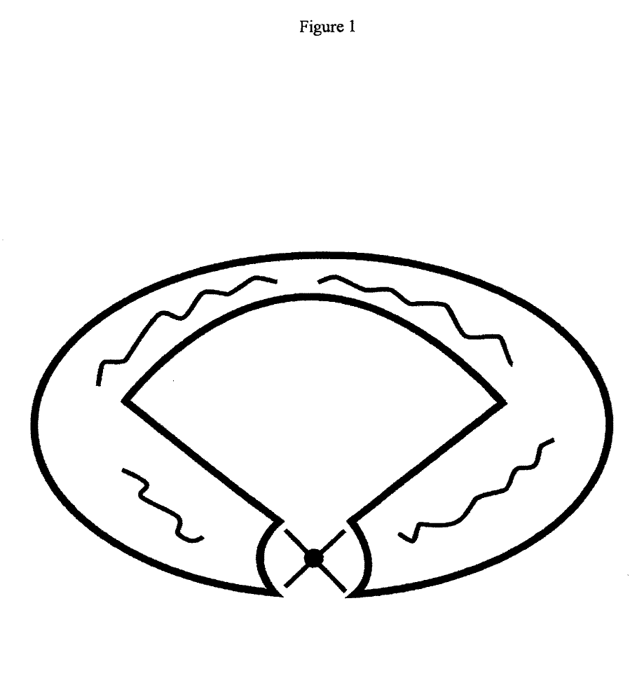

A first embodiment can be an ocular implant and surgical method of implantation. The ocular implant has features allowing a physician to re-fill the ocular implant without having to remove the implant from the area of implantation. Additionally, the implant can be constructed of temperature resistant material and encased in double insulated material with vacuum or air in between and microchannels with a pressure gradient and valves for egress of inherently cold or frozen material as shown in FIG. 1. The implantation technique is based upon creating a pocket within the eye.

The ocular implant is embedded with the drug to be delivered. The implant is comprised of a semi-porous polymer. The preferred embodiment is a variant of polyethylene glycol, but esters or other variants with cross linkage can be used as well as other semi-porous polymers obvious to one skilled within the art. The ocular implant should be of a semi-hard sponge-like consistency so that the implant can be re-loaded with the drug of choice by a needle injection. This consistency also allows the drug to be easily released within the eye at a desired location. The implant is shaped to fit within the pocket created by the surgical technique. The preferred shape is to fit within a scleral pocket and rest up against the scleral bed. The ocular implant is also coated with a water tight silicone coating on areas not in contact with the bed of the eye within the pocket. The preferred coating of the ocular implant is on three sides leaving the area of contact within the scleral bed uncoated for drug delivery.

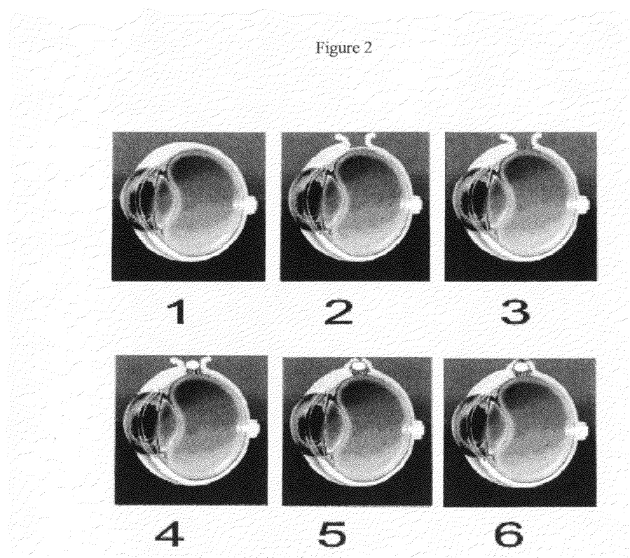

The surgical technique for the intrascleral approach is shown in FIG. 2 and is comprised applying speculum on the lids of the eye (1), forming a scleral pocket by raising flaps in the scleral bed where the scleral flaps are raised using a partial thickness cut as described in Schepens, Charles L. Retinal Detachment and Allied Diseases (2 vols). Philadelphia, Saunders, 1983 (2^{nd} edition with Mary E. Hartnett and Tastuo Hirose. Boston, Butterworth Heinemann, 2000). (2), creating a chorioretinal scar such as by applying diathermy with a designated path—as detailed above, (3), inserting the ocular implant soaked in the desired drug into the pocket (4), re-approximating the scleral flaps over the implant (5), and closing the pocket with a suture (6).

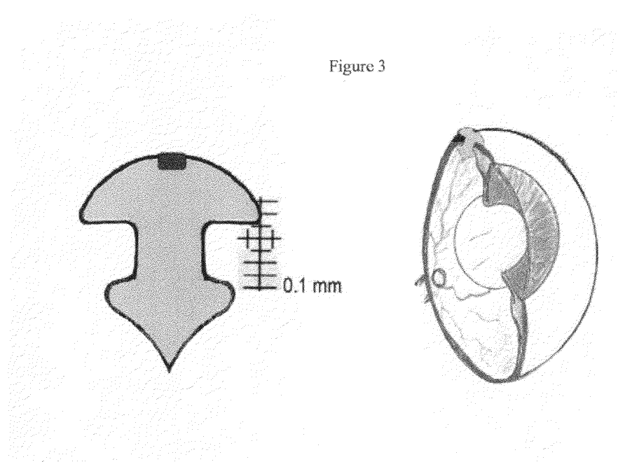

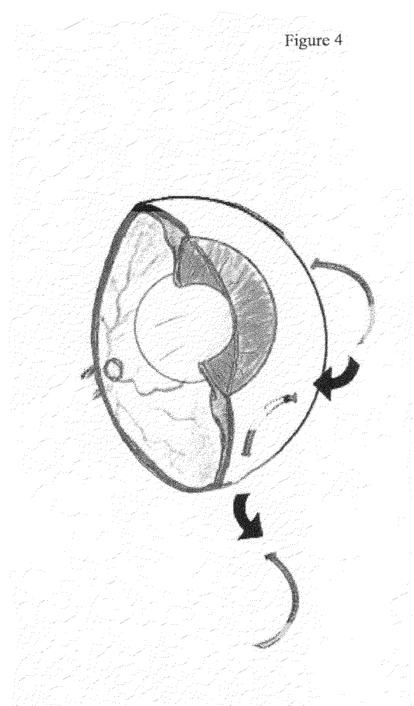

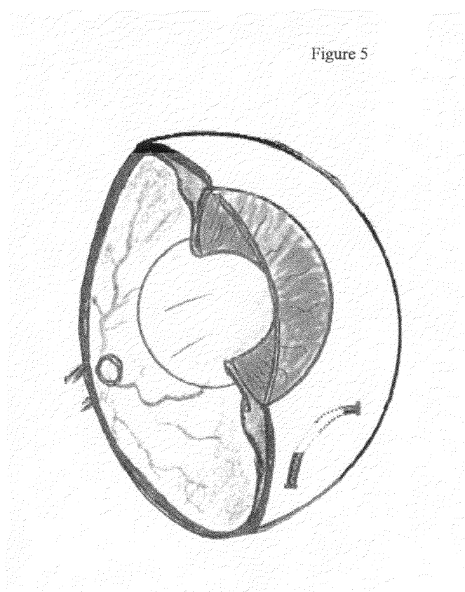

The surgical technique for a transcleral implant comprises a pin, a suture, and a loaded haptic. A self retaining pin can be shaped with an attachment end which contains an end point for attachment in the eye which widens and then narrows for insertion and attachment in the eye as in FIG. 3 can be made of a solid polymer shell with a refillable interior with a diffusion membrane on the lower side. The pin can be implanted after performing limbal peritomy and is implanted simply by pushing the self retaining pin in the pars plana as shown in FIG. 3 part B. The loaded haptic is an IOL with drugs imbedded in the polymer. A suture guided implant could also be used (FIG. 4). The suture guided implant can utilize a limbal peritomy in any quadrant then the suture-guided system can be passed in the pars plana until the distal end reservoir reaches the sclera (FIG. 4, step 1). The P.E. tube can be cut after it exits the sclera at a predetermined sealed point (FIG. 4, step 2). The P.E. can then be flattened past the pre-placed sealed point (FIG. 4, step 3). The loaded P.E is retrained within the eye with sealed edges. (FIG. 5).

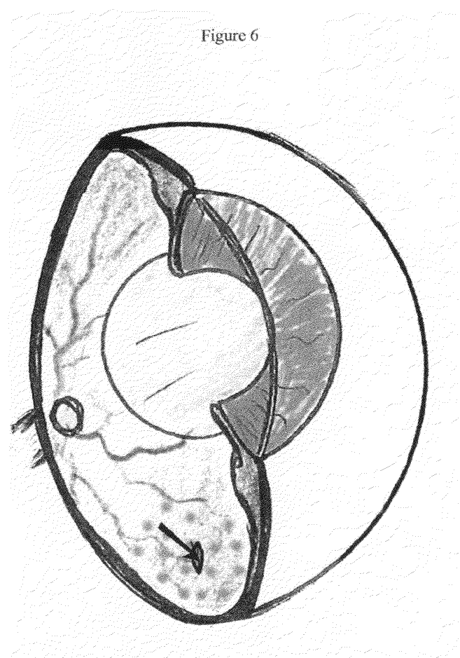

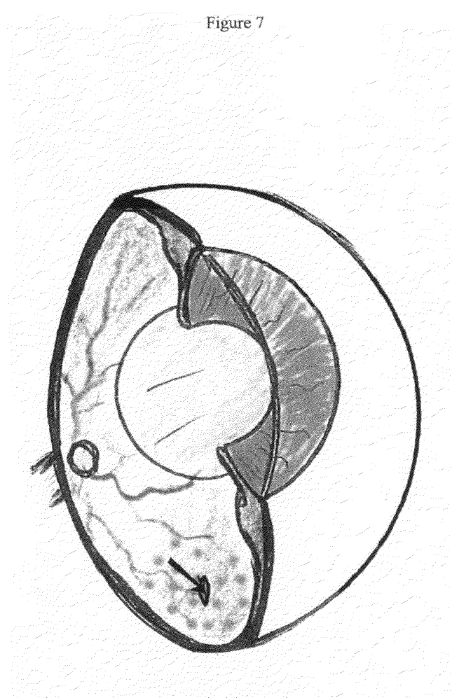

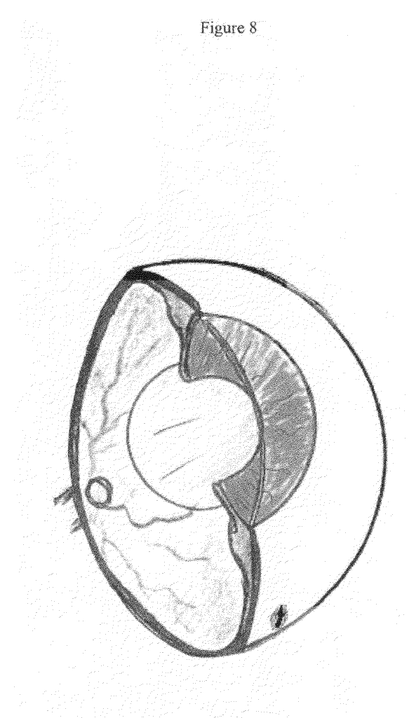

The surgical technique for subretinal implant systems comprises a transvitrial needle subretinal delivery of a viscous polymer delivered through the pars plana via a long self sealing 27G needle to the subretinal space as shown in FIG. 6. A Pars Plana Vitrectomy (PPV) approach for subretinal implantation could utilize a conventional PPV followed by retinotomy, surrounded by laser, then the delivery of fluid, semi-solid or solid implant as shown in FIG. 7. A posterior scleral approach as shown in FIG. 8 after laser or diathermy pre-treatment could be comprised of a posterior sclerotomy (similar to that used for drainage) and the dissection of the potential suprachoroidal space and the injection or insertion of an implant of fluid, semi-solid, or solid in the pretreated area.

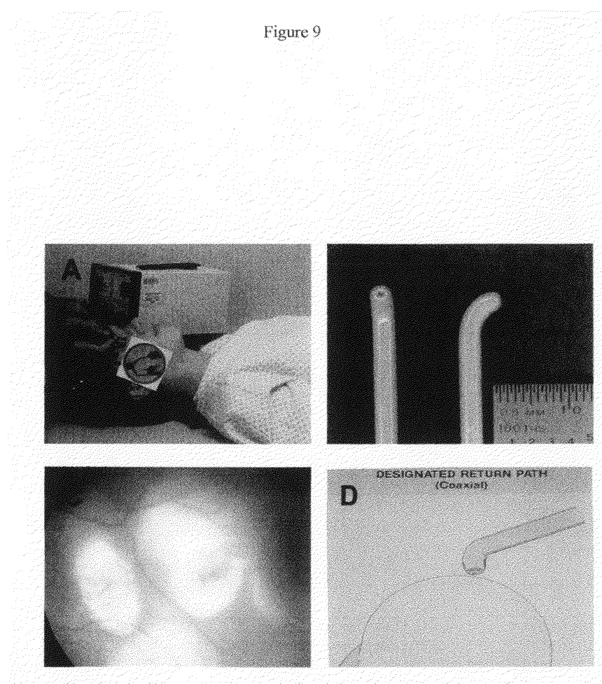

An embodiment of implied diathermy utilizes a co-axial delivery and designated return path to establish a closed and predictable bipolar circuit thus the resulting burn is safe and predictable. In an embodiment the technique for diathermy is to apply speculum between the eyelids, then apply an electrode to desired part of the eye through the speculum where the electrode is connected to the ground electrode of the diathermy circuit. For partial thickness inner scleral application, apply power until black scarring starts forming on the scleral bed, and then performing a grid pattern to cover the entire scleral bed. For trans-scleral application, perform the burn using an electrode such as the Jabbour electrode (FIG. 9) and simultaneously observe the retina with an indirect opthalmoscope so as to stop when the retina starts showing a white burn as described further below. The use of diathermy creates the changes in the layers needed for facilitating penetration. The Jabbour electrode (JE) has an effective width of about 16.7 to 27.7 about mm including an effective width of about 22.2 mm, an effective length of about 25.5 to about 42.5 mm including an effective length of about 34.0 mm, an effective curvature of about 1.5 to about 2.5 mm in length including an effective curvature of about 2.0 mm, and a rounded bent contact end of an effective curvature angle of about 105° to about 165° including an effective curvature of about 135° (FIG. 9B) that allows free sliding and indentation on the sclera without damage to the tissue. Due to the wide rounded bent tip shape, the JE distributes the diathermy energy deeper into the tissue, allowing for retinal burn through full thickness sclera, without scleral damage (proved by pathology). Also, due to its shape, the JE allows for simultaneous indirect opthalmoscopic observation to evaluate the end-point for the burn.

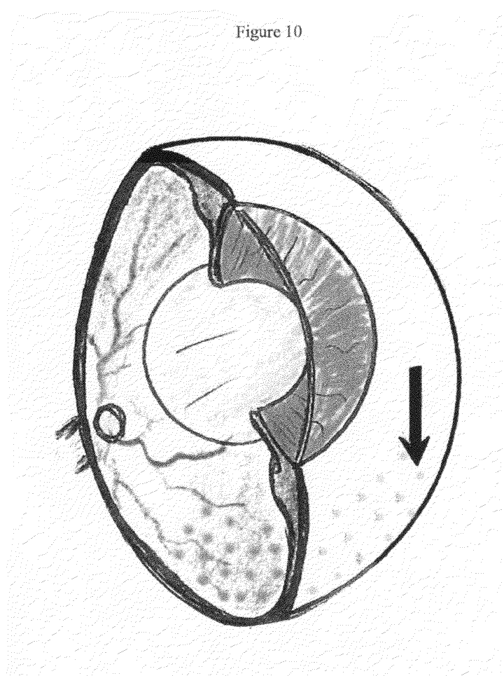

An additional embodiment of diathermy is the use of trans-conjunctival diathermy as shown in FIG. 9. During this technique the return path lid speculum (A) is inserted between the lids as in FIG. 9 then the Jabbour electrode (B) is applied on the conjunctiva while the retina is observed using indirect opthalmoscopy. Once the diathermy power creates a straw color burn (C) on the retina, additional applications are applied spaced 2 burn-widths apart as in FIG. 10 are made in each quadrant until each quadrant is filled with applications while avoiding the horizontal structures at 3:00 and 9:00 and a coaxial return path is used (D) as described below. Trans-conjunctival diathermy allows for eye drops to penetrate better and last longer. Also sub-tenon's or sub-conjunctival drugs either injected or implanted are able to penetrate better and last longer after the procedure. The use of injectable drugs formulated as eye drops and gels can be used externally but with good penetration due to the trans-conjunctival diathermy

The technique for reloading the ocular implant is the same for all techniques using an ocular implant and comprises of loading a syringe with the desired drug and then releasing the drug into the ocular implant through an external injection through the silicone coating of the ocular implant, using a sharp needle

No matter the method of implantation used, long-term implants for protein-based agents can be incased in double insulated material with vacuum or air insulation and microchannels with a pressure gradient and valves used for egress of inherently cold or frozen material.

The intraocular depot comprises inserting a liquid depot that will become semi-gelatinous in consistency at 37° C. and sink to the bottom of the vitreous.

These terms and specifications, including the examples, serve to describe the invention by example and not to limit the invention. It is expected that others will perceive differences, which, while differing from the forgoing, do not depart from the scope of the invention herein described and claimed. In particular, any of the function elements described herein may be replaced by any other known element having an equivalent function.

What is claimed is:

1. A method comprising a bipolar diathermy for the eye wherein an electrode is connected to the ground electrode of the diathermy circuit, the electrode is then applied to the desired parts of the eye through a speculum applied between the eyelids to reach the eye, and power is delivered to the electrode to create changes in layers in the desired part of the eye using a designated, co-axial delivery and return path from the eye.

2. The method of claim 1 wherein the desired part of the eye is the inner sclera wherein the electrode is applied to the sclera, power is applied to the electrode until black scarring starts forming on the inner scleral bed, and a grid pattern is performed to cover the entire scleral bed.

3. The method of claim 2 wherein the electrode is a Jabbour electrode.

4. The method of claim 1 wherein the desired part of the eye is the trans-sclera wherein the electrode is applied to the trans-sclera and power is added to cause a burn to the retina while the retina is observed with an indirect opthalmoscope so as to stop when the retina starts showing a white burn and additional applications are applied spaced 2 burn-widths apart in each quadrant until each quadrant is filled with applications while avoiding the horizontal structures at 3:00 and 9:00.

5. The method of claim 4 wherein the electrode is a Jabbour electrode.

6. The method of claim 1 wherein the desired part of the eye is the trans-conjunctiva wherein the electrode is applied on the conjunctive and power is added while the retina is observed using indirect opthalmoscopy, power is added until a straw colored burn is observed on the retina, and additional applications are applied spaced 2 burn-widths apart in each quadrant until each quadrant is filled with applications while avoiding the horizontal structures at 3:00 and 9:00.

7. The method of claim 6 wherein the electrode is a Jabbour electrode.

8. The method of claim 1 wherein the desired part of the eye is the sclera and a scleral pocket is formed by raising flaps in the scleral bed by the electrode, a chorioretinal scar is created by the electrode, an ocular implant soaked in a desired drug is implanted in the pocket, the scleral flaps are re-approximated over the implant, and the pocket is closed with a suture.

9. An ocular implant comprising a semi-porous polymer of a semi-hard sponge-like consistency shaped to fit the scleral bed the ocular implant further comprised of a double insulated material with one of a vacuum or air insulation and microchannels with one or more pressure gradient and valves.

10. The ocular implant of claim 9 the ocular implant further comprised of water tight silicone coating on all areas not to be in contact with the bed of the inner scleral within the pocket.

11. The ocular implant of claim 9 wherein the semi-porous polymer is polyethylene glycol.

12. The ocular implant of claim 9 wherein the ocular implant is a self retaining pin.

13. The ocular implant of claim 12 wherein the ocular implant is implanted through the sclera after a limbal peritomy is performed.

14. The ocular implant of claim 12 wherein the ocular implant is implanted through the sclera after a limbal peritomy is performed and a suture-guided system can be passed in the pars plana until the distal end reservoir reaches the sclera, the P.E. tube is cut after exiting the sclera at a predetermined sealed point, the P.E. is flattened past the pre-placed sealed point, and the loaded P.E. is retained within the eye with sealed edges, outside the sclera and under the conjunctiva.

15. A diathermy electrode comprising a contact end with an effective width, an effective length, an effective length of curvature, and an effective curvature angle for free sliding and indentation of the sclera without damage to the scleral tissue.

16. The diathermy electrode of claim 15 wherein the effective width is about 16.7 to 27.7 about mm.

17. The diathermy electrode of claim 15 wherein the effective length is about 25.5 to about 42.5 mm.

18. The diathermy electrode of claim 15 wherein the effective curvature is about 1.5 to about 2.5 mm in length.

19. The diathermy electrode of claim 15 where in the effective curvature angle is about 105° to about 165°
